(19) United States
(12) Patent Application Publication
(10) Pub. No.: US 2026/0306576 A1
(43) Pub. Date: Oct. 8, 2026

(54) COMPOSITIONS AND METHODS FOR APPETITE SUPPRESSION AND IMPROVING METABOLIC HEALTH

(71) Applicant: BioTRUST Nutrition, LLC, Littleton, CO (US)

(72) Inventors: Timothy Michael Skwiat, Austin, TX (US); Andrew J. Quintanilla, Schertz, TX (US)

(73) Assignee: BioTRUST Nutrition, LLC, Littleton, CO (US)

(21) Appl. No.: 19/641,958

(22) Filed: Apr. 8, 2026

Related U.S. Application Data
(60) Provisional application No. 63/785,342, filed on Apr. 8, 2025.

(51) Int. Cl. A61K 36/87; A61K 36/185; A61K 36/82; A61K 36/85

(52) U.S. Cl. CPC A61K 36/87; A61K 36/5775; A61K 36/82; A61K 36/85

(57) ABSTRACT

Provided herein are compositions for use as dietary supplements. The disclosed dietary supplements comprise essentially natural ingredients, one or more of which promote healthy weight management and metabolic wellness.

### CROSS REFERENCE TO RELATED APPLICATION

This application claims priority to U.S. Provisional Patent Application Ser. No. 63/785,342, filed Apr. 8, 2025, and titled “COMPOSITIONS AND METHODS FOR APPETITE SUPPRESSION AND IMPROVING METABOLIC HEALTH,” which is incorporated by reference herein in its entirety.

### BACKGROUND

### 1. Field

The present disclosure broadly relates to compositions for use as dietary supplements for weight loss and improving metabolic health.

### 2. Discussion of Related Art

Obesity is a major public health concern, affecting over 1 billion people worldwide, including 650 million adults and over 150 million children and adolescents. In the United States, more than 42% of adults are classified as obese, a figure that has risen dramatically in recent decades. Obesity is linked to severe health complications, including type 2 diabetes, cardiovascular disease, and certain cancers, leading to an increased risk of premature death. In addition to personal health impacts, obesity places a significant burden on healthcare systems, with annual obesity-related costs in the U.S. exceeding $170 billion.

Glucagon-like peptide-1 (GLP-1) receptor agonists have emerged as a breakthrough in the management of obesity and metabolic health. Originally developed to treat type 2 diabetes, GLP-1 medications have demonstrated significant weight loss benefits by mimicking the natural GLP-1 hormone, which regulates blood sugar levels and appetite. By enhancing insulin secretion, slowing gastric emptying, and increasing satiety, these drugs help individuals reduce caloric intake and sustain long-term weight loss.

While GLP-1 receptor agonists, such as semaglutide (Wegovy®, Ozempic®) and tirzepatide (Mounjaro®, Zepbound®), have demonstrated remarkable efficacy in reducing body weight there is growing interest in natural ways to boost GLP-1 as a safer and more sustainable alternative. Unlike pharmaceutical interventions, which can cause side effects such as nausea, vomiting, and gastrointestinal discomfort, natural methods work with the body's own mechanisms to enhance GLP-1 secretion. These approaches not only support appetite control but also improve overall metabolic health without the potential risks associated with long-term medication use. There is therefore a need for developing natural GLP-1 boosters.

### SUMMARY

In some aspects, the current disclosure encompasses a dietary supplement comprising: Hibiscus sabdariffa (rosella) flower extract; Lippia citriodora (lemon verbena) leaf extract; Cissus quadrangularis whole plant extract; a polyphenol blend; and at least one excipient. In some aspects, the dietary supplement comprises at least, about, or at most 1-10% by weight of rosella flower extract, and at least, about, or at most 1-30% by weight lemon verbena leaves extract. In some aspects, the Hibiscus sabdariffa flower and the Lippia citriodora leaf extracts are present in a 2:0.5 to 0.5:2 ratio by weight. In some aspects, the dietary supplement comprises Metabolaid®. In some aspects the polyphenol blend provides at least, about, or at most 100-300 mg of polyphenols per 1000 mg of the dietary supplement excluding excipients. In some aspects, the polyphenol blend comprises green tea (Camellia sinensis), pomegranate (Punica granatum), and grape (Vitis vinifera) whole plant extract. In some aspects, at least one excipient comprises one or more diluents, stabilizers, antioxidants, fillers, carriers, adjuvants, lubricant, preserving agents, stabilizing agents, wetting agents, emulsifying agents, solution promoters, salts, solubilizers, antifoaming agents, antioxidants, dispersing agents, surfactants, or colorants, or any combination thereof. In some aspects, the filler is rice flour. In some aspects, the carrier is hypromellose. In some aspects, the dispersing agent is silicon dioxide. In some aspects, the lubricant is magnesium stearate. In some aspects, the dietary supplement comprises about 300-700 mg combined of Hibiscus sabdariffa and Lippia citriodora extracts, about 200-400 mg of Cissus quadrangularis extract, and about 200-400 mg polyphenol blend. In some aspects, the dietary supplement comprises about 500 mg by weight of Hibiscus sabdariffa flower and Lippia citriodora leaf extract; about 300 mg by weight of Cissus quadrangularis extract; about 200 mg by weight of polyphenol blend; and at least one excipient.

In some aspects, the dietary supplement is in the form of a solid, a semi-solid, or a liquid. In some aspects, the dietary supplement is in the form of a tablet, a capsule, or a gummy. In some aspects, the dietary supplement is free from artificial colors and preservatives. In some aspects, the dietary supplement is gluten free, soy free, nut free, and/or dairy free. In some aspects, the dietary supplement is vegan.

In some aspects, administering the dietary supplement to a subject in need thereof, enhances the production of glucagon-like peptide 1 (GLP-1) in the subject. In some aspects, administering the dietary supplement to a subject in need thereof, reduces the degradation of glucagon-like peptide 1 (GLP-1) in the subject. In some aspects, administering the dietary supplement to a subject in need thereof, increases the overall levels of GLP-1 by over 30% in the first 8 weeks of daily administration of 500 mg of Metabolaid®, 300 mg of Cissus quadrangularis extract and 200 mg of the polyphenol blend per 1000 mg of the dietary supplement excluding excipients. In some aspects, the subject is a human.

Also, disclosed herein is a method of managing weight and/or improving metabolic health in a subject in need thereof, the method comprising administering to the subject a dietary supplement comprising: one or more sources of anthocyanins; one or more sources of phenylpropanoids; a polyphenol blend; and at least one excipient. In some aspects, the method comprises administering the dietary supplement in an amount that provides about 500 mg of Metabolaid®, 300 mg of Cissus quadrangularis extract and 200 mg of the polyphenol blend daily, at least 15-45 minutes before a meal. In some aspects, the amount is administered in a single dose, 2 doses, or more. In some aspects, the method comprises administering the dietary supplement orally.

In some aspects of the method, administering the dietary supplement to a subject in need thereof, enhances the production of glucagon-like peptide 1 (GLP-1) in the subject. In some aspects, administering the dietary supplement to a subject in need thereof, reduces the degradation of glucagon-like peptide 1 (GLP-1) in the subject. In some aspects, administering the dietary supplement to a subject in need thereof, increases the overall levels of GLP-1 by over 30% in the first 8 weeks of daily.

Also disclosed herein, is a method of administering a dietary supplement to a subject, the method comprising, (a) providing a dietary supplement as disclosed herein; and (b) instructing the subject to take 2 capsules daily, 30 minutes before a meal.

### DETAILED DESCRIPTION

The following detailed description references the accompanying drawings that illustrate various aspects of the present disclosure. The drawings and description are intended to describe aspects of the present disclosure in sufficient detail to enable those skilled in the art to practice the present disclosure. Other components can be utilized, and changes can be made without departing from the scope of the present disclosure. The following description is, therefore, not to be taken in a limiting sense.

In some aspects, the present disclosure encompasses dietary supplements formulated to support healthy appetite control and overall metabolic wellness. The compositions may help maintain normal satiety responses and support balanced energy use through naturally derived plant extracts. In certain embodiments, the ingredients may help promote a healthy digestive environment, including supporting beneficial gut microbial activity and the natural production of short-chain fatty acids. The disclosed formulations may further support feelings of fullness, healthy eating patterns, and general metabolic balance, including supporting the body's natural processes involved in carbohydrate and fat utilization. In some aspects, the supplement may also help maintain already-healthy blood sugar levels and contribute to overall healthy body composition when used as part of a balanced lifestyle.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface.

The terms “comprising,” “including,” and “having” are used interchangeably in this disclosure. The terms “comprising,” “including,” and “having” mean to include, but not necessarily be limited to the things so described.

The terms “or” and “and/or” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B, and C.” An exception to this definition will occur only when a combination of elements, functions, steps, or acts are in some way inherently mutually exclusive.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (3rd ed. 2006); Chambers Dictionary of Science and Technology (Walker ed., 1999); The Glossary of Genetics, 5th Ed., R. Rieger et al. (2008), The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

As used herein, “supporting,” “promoting,” and/or “maintaining” refer to the use of dietary supplements to support the body's normal physiological functions, including but not limited to metabolic processes, appetite regulation, and overall wellness. These terms are not intended to imply the diagnosis, treatment, cure, or prevention of any disease.

As used herein, “support” or “maintenance” refers to aiding the body's natural ability to maintain normal physiological functions, including healthy metabolic processes, appetite regulation, and body composition, as part of a balanced lifestyle.

As used herein, “individual,” “subject,” “host,” and “patient” can be used interchangeably herein and refer to any mammalian subject for whom diagnosis, treatment, prophylaxis or therapy is desired, for example, humans, pets, livestock, horses or other animals. As used herein, the term “subject” and “patient” are used interchangeably herein and refer to both human and nonhuman animals. The term “non-human animals” of the disclosure includes all vertebrates, e.g., mammals and non-mammals, such as nonhuman primates, sheep, dog, cat, horse, cow, chickens, amphibians, reptiles, and the like. In some aspects, the subject can be a human.

### II. Active Ingredients

In some aspects, the present invention is premised on a determination that administration of a combination of one or more of active ingredients including one or more sources of anthocyanins, one or more sources of verbascoside, one or more sources of ketosteroids, one or more sources of ellagic acid, and one or more sources of polyphenols can be used to bring about appetite suppression and improve metabolic health. Provided herein are compositions for use as dietary supplements for appetite suppression and for improving metabolic health.

In some aspects, the dietary supplements disclosed herein comprise about 1%-10% by weight in anthocyanins. In some aspects, the dietary supplement comprises at least, at most, exactly, or about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50%, or any range derivable therein of anthocyanins.

Non-limiting examples of natural sources of anthocyanin include Vaccinium corymbosum (blueberries), Rubus fruticosus (blackberries), Rubus idaeus (raspberries), Prunus avium (cherries), Ribes nigrum (blackcurrants), Brassica oleracea var. capitata f. rubra (red cabbage), Ipomoea batatas (purple sweet potatoes), Solanum melongena (eggplant skin), Vitis vinifera (grapes), Prunus domestica (plums), Fragaria ananassa (strawberries), Sambucus nigra (elderberries), Punica granatum (pomegranates), Citrus sinensis (blood oranges), Hibiscus sabdariffa (hibiscus), Viola tricolor (pansies), Petunia spp. (petunias), Rosa spp. (roses), and Centaurea cyanus (blue cornflowers). In some aspects, the disclosed dietary supplement comprises one or more sources of anthocyanin. In some aspects, the one or more sources of anthocyanin comprise Hibiscus sabdariffa (hibiscus).

In some aspects, the dietary supplements disclosed herein comprise about 5%-30% by weight in phenylpropanoids. In some aspects, the dietary supplement comprises at least, at most, exactly, or about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50% or any range derivable therein of phenylpropanoids. In some aspects, the dietary supplements disclosed herein comprise about 1%-10% by weight in polyphenols. In some aspects, the dietary supplement comprises at least, at most, exactly, or about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50%, or any range derivable therein of polyphenols.

Non-limiting examples of natural sources of phenylpropanoids include Cinnamomum verum (cinnamon), Coffea arabica (coffee), Salvia officinalis (sage), Mentha piperita (peppermint), Echinacea purpurea (purple coneflower), Allium sativum (garlic), Hordeum vulgare (barley), Vitis vinifera (grapes), Capsicum annuum (bell pepper), Thymus vulgaris (thyme), Rosmarinus officinalis (rosemary), Matricaria chamomilla (chamomile), Ocimum basilicum (basil), Cucumis sativus (cucumber), and Lippia citriodora (lemon verbena). In some aspects, the disclosed dietary supplement comprises one or more sources of phenylpropanoids. In some aspects, the one or more sources of anthocyanin comprise Lippia citriodora (lemon verbena).

Non-limiting examples of natural sources of polyphenols include Coffea arabica (coffee), Camellia sinensis (tea), Vitis vinifera (grapes), Malus domestica (apples), Vaccinium corymbosum (blueberries), Rubus idaeus (raspberries), Prunus domestica (plums), Fragaria ananassa (strawberries), Theobroma cacao (cocoa), Allium sativum (garlic), Cinnamomum verum (cinnamon), Capsicum annuum (bell pepper), Triticum aestivum (wheat), Punica granatum (pomegranate), Spinacia oleracea (spinach), Rosa canina (rose hips), Mentha piperita (peppermint), and Zea mays (corn). In some aspects, the disclosed composition comprises one or more sources of polyphenols. In some aspects, the one or more sources of polyphenols comprise green tea (Camellia sinensis), pomegranate (Punica granatum), or grape (Vitis vinifera) extract, or any combination thereof.

In some aspects, the dietary supplements disclosed herein comprise about 5%-30% by weight in ketosteroids. In some aspects, the dietary supplement comprises at least, at most, exactly, or about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50%, or any range derivable therein of ketosteroids.

Non-limiting examples of natural sources of ketosteroids include Cissus Quadrangularis, Withania somnifera (ashwagandha), and Nicandia physalodes (apple of Peru) extract, or any combination thereof.

In some aspects, one or more of the active ingredients of the disclosed dietary supplement are derived from non-genetically modified (non-GMO) sources. In some aspects, all the active ingredients of the disclosed dietary supplement are derived from non-GMO sources. In some aspects the dietary supplements disclosed herein do not comprise any ingredients that contain wheat, gluten, soy, dairy products and/or nuts.

In some aspects, the dietary supplements of the current disclosure comprise one or more active ingredients, for example extracts from Hibiscus sabdariffa, Lippia citriodora, Cissus quadrangularis, and/or polyphenol extract.

1. Hibiscus sabdariffa (Roselle, Red Sorrel, Jamaican Sorrel, Indian Sorrel, Guinea Sorrel)

Hibiscus sabdariffa commonly known as roselle or hibiscus, is a flowering plant widely consumed as herbal tea and valued for its potential metabolic health benefits. Rich in polyphenols, anthocyanins, and flavonoids, in some aspects, hibiscus may support weight management, improve metabolic health, and help maintain healthy blood sugar levels. In some aspects, the dietary supplement disclosed herein may comprise at least, at most, or about 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, or 25%, or any range derivable therein of Hibiscus sabdariffa extract. In some aspects, the extract is derived from Hibiscus sabdariffa flowers. In some aspects, the dietary supplement comprises about 100 mg-300 mg of Hibiscus sabdariffa flower extract per 1000 mg of the dietary supplement. In some aspects, the dietary supplement comprises a commercially available formulation of Hibiscus sabdariffa extract, for example, METABOLAID® in an amount of about 300 mg, 400 mg, 500 mg, 600 mg, to 700 mg per 1000 mg of the dietary supplement excluding excipients. In some aspects, the dietary supplement comprises a commercially available formulation of Hibiscus sabdariffa extract, for example, METABOLAID® in an amount of about 500 mg, per 1000 mg of the dietary supplement excluding excipients.

2. Lippia citriodora

Lippia citriodora, commonly known as lemon verbena, contains significant levels of phenylpropanoids, which contribute to its metabolic health benefits. In some aspects, the dietary supplement disclosed herein may comprise at least, at most, or about 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50%, or any range derivable therein, of Lippia citriodora extract. In some aspects, the extract is derived from Lippia citriodora leaves. In some aspects, the dietary supplement comprises about 200 mg-400 mg of Lippia citriodora leaves extract per 1000 mg of the dietary supplement. In some aspects, the dietary supplement comprises a commercially available formulation of Lippia citriodora extract, for example, METABOLAID® in an amount of about 300 mg, 400 mg, 500 mg, 600 mg, to 700 mg per 1000 mg of the dietary supplement excluding excipients. In some aspects, the dietary supplement comprises a commercially available formulation of Lippia citriodora extract, for example, METABOLAID® in an amount of about 500 mg, per 1000 mg of the dietary supplement excluding excipients.

### 3. Cissus Quadrangularis

Cissus Quadrangularis (additional names and sources: Asthisonhara, Chadhuri, Chaudhari, Cissus, Cissus Extract, Cissus Formula, Cissus Formulation, C. Quadrangularis, CORE, CQ, CQE, CQR-300, Extrait de Cissus, Hadjod, Hadjora, Harbhanga, Harsankari, Hasjora, Kandavela, Mangaroli, Nalleru, Namunungwa, Phet Cha Sung Khaat, Phet Sang Kat, Phet Sangkhat, Pirandai, Quadrangularis, Samroi To, San Cha Khuat, Stemmed Vine, Vajravalli, Vedhari, Veld Grape, Veldt-grape, Vitis quadrangularis, Winged Treebine) is a tropical plant traditionally used in Ayurvedic medicine for its various health benefits, including promoting metabolic health. In some aspects, the dietary supplement disclosed herein may comprise at least, at most, or about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, or 25%, or any range derivable therein, of Cissus Quadrangularis extract. In some aspects, the extract is a whole plant extract. In some aspects, the dietary supplement comprises about 100 mg-300 mg of Cissus Quadrangularis extract per 1000 mg of the dietary supplement. In some aspects, the dietary supplement comprises a commercial formulation of Cissus Quadrangularis extract, for example CQR-300® in an amount of about 100 mg, 200 mg, 300 mg, 400 mg, 500 mg per 1000 mg of the dietary supplement excluding excipients. In some aspects, the dietary supplement comprises a commercial formulation of Cissus Quadrangularis extract, for example CQR-300® in an amount of 300 mg per 1000 mg of the dietary supplement excluding excipients.

### 4. Polyphenol Blend

In some aspects, the dietary supplements disclosed herein further comprises a polyphenol blend. In some aspects, the polyphenol blend comprises one or more of green tea (Camellia sinensis), pomegranate (Punica granatum), grape (Vitis vinifera) whole plant extract, or any combination thereof.

Green Tea (Camellia sinensis)

Camellia sinensis, commonly known as green tea, is recognized for its beneficial effects on metabolic health. Rich in polyphenols, particularly catechins such as epigallocatechin gallate (EGCG), green tea may enhance fat metabolism, improve insulin sensitivity, and support weight management. In some aspects and without being bound by theory, the EGCG and caffeine in green tea may work synergistically to boost thermogenesis and fat oxidation. In some aspects, the dietary supplement disclosed herein may comprise at least, at most, or about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, or 25% of Camellia sinensis extract. In some aspects, the extract is a whole plant extract. In some aspects, the dietary supplement comprises about 10 mg-100 mg of Camellia sinensis extract per 1000 mg of the dietary supplement. In some aspects, the dietary supplement comprises 100 mg of Camellia sinensis extract per 1000 mg of the dietary supplement.

Pomegranate (Punica granatum)

Punica granatum, commonly known as pomegranate, is a nutrient-rich fruit with potent bioactive compounds that may support metabolic health. It has multiple polyphenols, particularly ellagitannins, anthocyanins, and flavonoids, which exhibit strong antioxidant and anti-inflammatory properties. Some traditional names for pomegranate include Anar (Hindi, Persian), Granada (Spanish), and Rumman (Arabic). In some aspects, the dietary supplement disclosed herein may comprise at least, at most, or about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, or 25% of Punica granatum extract. In some aspects, the extract is a whole plant extract. In some aspects, the dietary supplement comprises about 10 mg-100 mg of Punica granatum extract per 1000 mg of the dietary supplement. In some aspects, the dietary supplement comprises about 10 mg-100 mg of Punica granatum extract per 1000 mg of the dietary supplement.

Grape (Vitis vinifera)

Vitis vinifera, commonly known as grape, is a rich source of polyphenols, including resveratrol, flavonoids, and anthocyanins, which may contribute to metabolic health. With their strong antioxidant properties, grapes help combat oxidative stress, a major factor in metabolic dysfunction. Some alternate names for grapes include Angoor (Hindi, Urdu), and Uva (Spanish, Italian). In some aspects, the dietary supplement disclosed herein may comprise at least, at most, or about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, or 25% of Vitis vinifera whole or partial plant extract. In some aspects, the extract is a whole plant extract. In some aspects, the dietary supplement comprises about 10 mg-100 mg of Vitis vinifera whole or partial plant extract per 1000 mg of the dietary supplement. In some aspects, the dietary supplement comprises 50 mg of Vitis vinifera whole or partial extract per 1000 mg of the dietary supplement

In some aspects, the one or more active ingredients of the disclosed dietary supplement may be provided as a commercially available formulation (e.g., METABOLAID® as a source of Hibiscus sabdariffa and Lippia citriodora extracts; CQR-300® as a source of Cissus Quadrangularis extract) which may be combined with the rest of the ingredients. In some aspects, the dietary supplement comprises METABOLAID® in an amount of about 300 mg, 400 mg, 500 mg, 600 mg, to 700 mg per 1000 mg of the dietary supplement excluding excipients. In some aspects, the dietary supplement comprises CQR-300® in an amount of about 100 mg, 200 mg, 300 mg, 400 mg, or 500 mg per 1000 mg of the dietary supplement excluding excipients. In some aspects, the dietary supplements comprise 500 mg of METABOLAID® and/or 300 mg of CQR-300® per 1000 mg of the dietary supplement excluding excipients.

The amount of active substance in the disclosed dietary supplements or preparations is such that a suitable dosage in the range indicated is obtained. The compositions are preferably formulated in a unit dosage form, each dosage containing from about 1-1000 mg, 2-800 mg, 5-500 mg, 10-400 mg, 50-200 mg, e.g., about 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 200 mg, 300 mg, 400 mg, 500 mg, 600 mg, 700 mg, 800 mg, 900 mg, or 1000 mg of the active ingredient. The term “unit dosage form” refers to physically discrete units suitable as unitary (i.e., single) dosages for human subjects and other mammals, each unit containing a predetermined quantity of active material calculated to produce the desired effect, in association with a suitable excipient.

With this backdrop in mind, the current disclosure provides a unique combination of ingredients that are not only compatible within one another but actually work in a synergistic manner in improving metabolic health and suppress appetite. Even though individual health benefits of each of these ingredients have been explored, the studies are not all conclusive. Given the ambiguous effects of each of these plant extracts taken separately, it was highly surprising and unexpected that together these extracts can have such a significant impact on metabolic health and appetite as further illustrated in the examples.

### III. Excipients

In some aspects, dietary supplements disclosed herein may comprise one or more excipients suitable for use in dietary supplements. In some aspects, dietary supplements herein may comprise diluents, stabilizers, antioxidants, fillers, carriers, adjuvants, lubricant, preserving agents, stabilizing agents, wetting agents, emulsifying agents, solution promoters, salts, solubilizers, antifoaming agents, antioxidants, dispersing agents, surfactants, or colorants, or any combination thereof. Techniques for formulation and administration of drugs may be found in “Remington's Pharmaceutical Sciences,” Mack Publishing Co., Easton, Pa., latest edition, which is incorporated herein by reference.

In some aspects, the dietary supplement disclosed herein comprise at least, at most, about, or exactly 5-500 mg, 10-400 mg, 50-200 mg, e.g., 20 mg, 25 mg, 30 mg, 35 mg, 40 mg, 45 mg, 50 mg, 60 mg, 70 mg, 80 mg, 90 mg, 100 mg, 200 mg, 300 mg, 400 mg, 500 mg of total excipients per 1000 mg. In some aspects, the dietary supplement disclosed herein comprise about 250 mg of excipient per 1000 mg, or about 200 mg of excipient per 700 mg of the dry weight composition excluding the shell, for example a capsule shell.

In some aspects, to prepare the disclosed dietary supplements, the one or more active ingredients disclosed above are mixed with a suitable carrier. Upon mixing or addition of the compound(s), the resulting mixture may be a powder, solution, suspension, emulsion, or the like. The form of the resulting mixture depends upon a number of factors, including the intended mode of administration and the solubility of the compound in the selected carrier or vehicle. Carriers or vehicles suitable for administration of the dietary supplements provided herein include any such carriers known to be suitable for oral administration. In addition, the active materials can also be mixed with other active materials that do not impair the desired action, or with materials that supplement the desired action, or have another action.

In certain aspects, dietary supplements disclosed herein may comprise agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, dietary supplements disclosed herein may comprise at least 5%, at least 10%, at least 20%, at least 25%, at least 30%, at least 35%, at least 40%, at least 45%, or at least 50% total amount of one or more agents by total weight of the dietary supplement. In some aspects, dietary supplements disclosed herein may comprise about 5% to about 99%, about 10%, about 95%, or about 15% to about 90% total amount of one or more agents by total weight of the dietary supplement. In some aspects, one or more of these agents may be added to improve the performance, efficacy, safety, shelf-life and/or other property of the dietary supplement of the present disclosure. In some aspects, additives may be biocompatible, without being harsh, abrasive, and/or allergenic.

In certain aspects, dietary supplements disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any suitable organic or inorganic acid may be used. In some aspects, dietary supplements disclosed herein may comprise at least 5%, at least 10%, at least 20%, at least 25%, at least 30%, at least 35%, at least 40%, at least 45%, at least 50% total amount of one or more acidifying agents by total weight of the dietary supplement. In some aspects, dietary supplements disclosed herein may comprise about 5% to about 99%, about 10%, about 95%, or about 15% to about 90% total amount of one or more acidifying agents by total weight of the dietary supplement.

In certain aspects, dietary supplements disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any suitable organic or inorganic base can be used. In some aspects, dietary supplements disclosed herein may comprise at least 5%, at least 10%, at least 20%, at least 25%, at least 30%, at least 35%, at least 40%, at least 45%, at least 50% total amount of one or more alkalizing agents by total weight of the dietary supplement. In some aspects, dietary supplements disclosed herein may comprise about 5% to about 99%, about 10%, about 95%, or about 15% to about 90% total amount of one or more alkalizing agents by total weight of the dietary supplement.

In certain aspects, dietary supplements disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite and other materials known to one of ordinary skill in the art. In some aspects, dietary supplements disclosed herein may comprise at least 5%, at least 10%, at least 20%, at least 25%, at least 30%, at least 35%, at least 40%, at least 45%, at least 50% total amount of one or more antioxidants by total weight of the dietary supplement. In some aspects, dietary supplements disclosed herein may comprise about 5% to about 99%, about 10%, about 95%, or about 15% to about 90% total amount of one or more antioxidants by total weight of the dietary supplement.

In certain aspects, dietary supplements disclosed herein may comprise a buffer system. As used herein, a “buffer system” is a dietary supplement comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any suitable organic or inorganic buffer can be used. In some aspects, dietary supplements disclosed herein may comprise at least 5%, at least 10%, at least 20%, at least 25%, at least 30%, at least 35%, at least 40%, at least 45%, at least 50% total amount of one or more buffering agents by total weight of the dietary supplement. In some aspects, dietary supplements disclosed herein may comprise about 5% to about 99%, about 10%, about 95%, or about 15% to about 90% total amount of one or more buffering agents by total weight of the dietary supplement.

In some aspects, the amount of one or more buffering agents may depend on the desired pH level of a dietary supplement. In some aspects, dietary supplements disclosed herein may have a pH of about 6 to about 9. In some aspects, dietary supplements disclosed herein may have a pH greater than about 8, greater than about 7.5, greater than about 7, greater than about 6.5, or greater than about 6.

In certain aspects, dietary supplements disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin® (methylparaben), Nipasol™ (propylparaben), isopropyl alcohol, and any combination thereof. In some aspects, any suitable preservative can be used. In some aspects, dietary supplements disclosed herein may comprise at least 5%, at least 10%, at least 20%, at least 25%, at least 30%, at least 35%, at least 40%, at least 45%, or at least 50% total amount of one or more preservatives by total weight of the dietary supplement. In some aspects, dietary supplements disclosed herein may comprise about 5% to about 99%, about 10%, about 95%, or about 15% to about 90% total amount of one or more preservatives by total weight of the dietary supplement.

In certain aspects, dietary supplements disclosed herein may comprise one or more surface-acting reagents or detergents. In some aspects, surface-acting reagents or detergents may be synthetic, natural, or semi-synthetic. In some aspects, dietary supplements disclosed herein may comprise anionic detergents, cationic detergents, zwitterionic detergents, ampholytic detergents, amphoteric detergents, nonionic detergents having a steroid skeleton, or a combination thereof. In some aspects, dietary supplements disclosed herein may comprise at least 5%, at least 10%, at least 20%, at least 25%, at least 30%, at least 35%, at least 40%, at least 45%, or at least 50% total amount of one or more surface-acting reagents or detergents by total weight of the dietary supplement. In some aspects, dietary supplements disclosed herein may comprise about 5% to about 99%, about 10%, about 95%, or about 15% to about 90% total amount of one or more surface-acting reagents or detergents by total weight of the dietary supplement.

In certain aspects, dietary supplements disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, dietary supplements disclosed herein may comprise at least 5%, at least 10%, at least 20%, at least 25%, at least 30%, at least 35%, at least 40%, at least 45%, or at least 50% total amount of one or more stabilizers by total weight of the dietary supplement. In some aspects, dietary supplements disclosed herein may comprise about 5% to about 99%, about 10%, about 95%, or about 15% to about 90% total amount of one or more stabilizers by total weight of the dietary supplement.

In some aspects, dietary supplements disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a dietary supplement may be expressed in milliosmoles per liter (mOsm/L). Osmolarity may be measured using methods commonly known in the art. In some aspects, a vapor pressure depression method is used to calculate the osmolarity of the dietary supplements disclosed herein. In some aspects, the amount of one or more tonicity agents comprising a dietary supplement disclosed herein may result in a dietary supplement osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some aspects, a dietary supplement herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some aspects, a dietary supplement described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some aspects, dietary supplements disclosed herein may comprise at least 5%, at least 10%, at least 20%, at least 25%, at least 30%, at least 35%, at least 40%, at least 45%, or at least 50% total amount of one or more tonicity modifiers by total weight of the dietary supplement. In some aspects, dietary supplements disclosed herein may comprise about 5% to about 99%, about 10%, about 95%, or about 15% to about 90% total amount of one or more tonicity modifiers by total weight of the dietary supplement.

In certain aspects, dietary supplements described herein may be an aqueous suspension comprising one or more polymers as suspending agents. In some aspects, polymers that may comprise dietary supplements described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some aspects, dietary supplements disclosed herein may comprise at least 5%, at least 10%, at least 20%, at least 25%, at least 30%, at least 35%, at least 40%, at least 45%, or at least 50% total amount of polymers as suspending agent(s) by total weight of the dietary supplement. In some aspects, dietary supplements disclosed herein may comprise about 5% to about 99%, about 10%, about 95%, or about 15% to about 90% total amount of polymers as suspending agent(s) by total weight of the dietary supplement.

In certain aspects, dietary supplements disclosed herein may comprise a viscous formulation. In some aspects, viscosity of dietary supplement herein may be increased by the addition of one or more gelling or thickening agents. In some aspects, dietary supplements disclosed herein may comprise one or more gelling or thickening agents in an amount to provide a sufficiently viscous formulation to remain on treated tissue. In some aspects, dietary supplements disclosed herein may comprise at least 5%, at least 10%, at least 20%, at least 25%, at least 30%, at least 35%, at least 40%, at least 45%, or at least 50% total amount of gelling or thickening agent(s) by total weight of the dietary supplement. In some aspects, dietary supplements disclosed herein may comprise about 5% to about 99%, about 10%, about 95%, or about 15% to about 90% total amount of gelling or thickening agent(s) by total weight of the dietary supplement. In some aspects, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, hypermellose, plullulan, pectin Ghatti gum, guar gum, hectorite, lactose, sucrose, maltodextrin, mannitol, sorbitol, honey, maize starch, wheat starch, rice starch, potato starch, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In some aspects, one or more of the excipients disclosed herein may be from an animal source, a vegetarian source, or a vegan source. In some aspects, the disclosed dietary supplements comprise or consists essentially of excipients from vegan and/or vegetarian friendly sources. In some aspects, the one or more of the excipients disclosed herein are derived from non-GMO sources. In some aspects, all the excipients disclosed herein are derived from non-GMO sources. In some aspects the dietary supplements disclosed herein do not comprise any ingredients that contain wheat, gluten, soy, dairy products and/or nuts. In some aspects, the dietary supplements disclosed herein do not comprise artificial colors and/or preservatives.

In certain aspects, the present disclosure provides dietary supplements comprising one or more active ingredients disclosed herein, formulated for one or more routes of administration, for example oral administration. In some aspects, the dietary supplements of the present disclosure may be manufactured by processes well known in the art, e.g., by means of conventional mixing, dissolving, granulating, dragee-making, levigating, emulsifying, encapsulating, entrapping or lyophilizing processes.

In certain aspects, dietary supplements for use in accordance with the present disclosure may be formulated in conventional manner using one or more physiologically acceptable carriers comprising excipients, which facilitate processing of the active ingredients into preparations. In some aspects, the disclosed dietary supplements may comprise one or more fillers, dispersing agents and/or lubricants. Non-limiting examples include microcrystalline cellulose, magnesium stearate, silicon dioxide, rice powder, maltodextrin, calcium carbonate, gelatin (or a vegetarian equivalent thereof), starch, stearic acid, vegetable gum, lactose, dicalcium phosphate, hydroxypropyl methylcellulose, glycerin, and potassium sorbate, or any combination thereof.

In some aspects, the dietary supplements are formulated as a solid preparation for oral administration. In some exemplary aspects, the dietary supplements are formulated as tablets, gel capsules, gummies, self-dissolving strips, powder, sustained-release capsules, and the like for oral administration. In some aspects, the dietary supplement for oral administration may comprise one or more excipient. Non-limiting examples include microcrystalline cellulose, magnesium stearate, silicon dioxide, rice powder, maltodextrin, calcium carbonate, gelatin (or a vegetarian equivalent thereof), starch, stearic acid, vegetable gum, lactose, dicalcium phosphate, hydroxypropyl methylcellulose, glycerin, and potassium sorbate, or any combination thereof. In some aspects, dietary supplements herein may comprise the active ingredient in a powder form for constitution with a suitable vehicle, e.g., sterile, pyrogen-free water-based solution, before use. For oral administration, the dietary supplement can be provided in a formulation that protects it from the acidic environment of the stomach. For example, the dietary supplement can be formulated in an enteric coating that maintains its integrity in the stomach and releases the active inhibitor in the intestine. The dietary supplement may also be formulated in combination with an antacid or other such ingredient. Oral dietary supplements generally include an inert diluent or an edible carrier and may be compressed into tablets or enclosed in gelatin capsules. For the purpose of oral administration, the dietary supplements can be incorporated with excipients and used in the form of tablets, capsules, or troches. Pharmaceutically compatible binding agents and adjuvant materials can be included as part of the dietary supplement.

In various aspects, the tablets, pills, capsules, troches, and the like can contain any of the following ingredients or equivalents thereof: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, or fruit flavoring.

In some aspects, one or more excipients disclosed herein may be replaced by vegetarian or vegan equivalent. For instance, non-limiting examples of vegan replacements for gelatin include hypermellose, HPMC (Hydroxypropyl Methylcellulose), pullulan, agar-agar, pectin, carrageenan, konjac, corn starch, or any combination thereof.

Dietary supplements suitable for use in context of the present disclosure may include dietary supplements wherein the active ingredients can be contained in an amount effective to achieve the intended purpose. In some aspects, an effective amount means an amount of active ingredients effective to support healthy appetite regulation, metabolic function, and/or body composition.

In some aspects, the supplement described herein, or the pharmaceutical can be enclosed in multiple or single dose containers. The enclosed dietary supplements can be provided in kits, for example, including component parts that can be assembled for use. A kit may include a plurality of containers, each container holding one or more unit dose of the one or more active agents. The containers are preferably adapted for the desired mode of administration, including, but not limited to tablets, gel capsules, sustained-release capsules, and the like for oral administration.

The active ingredient may be administered at once or may be divided into a number of smaller doses to be administered at intervals of time. Concentrations and dosage values may also vary with the severity of the condition to be alleviated. For any particular subject, specific dosage regimens can be adjusted over time according to the individual need and the judgment of the person administering or supervising the administration of the dietary supplements, and that the concentration ranges set forth herein are exemplary only and are not intended to limit the scope or practice of the claimed dietary supplements.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The dietary supplements can also be administered as a component of an elixir, suspension, syrup, wafer, medicated chewing gum or the like. A syrup may contain, in addition to the active dietary supplements, sucrose as a sweetening agent and certain preservatives, dyes and colorings, and flavors.

The dietary supplements can also be mixed with other active agents that do not impair the desired action, or with materials that supplement the desired action.

It is contemplated that the dietary supplements of the present disclosure can include any amount of the ingredients discussed in this specification. The dietary supplements can also include any number of combinations of additional ingredients described throughout this specification. The concentrations of any ingredient within the dietary supplements can vary. In non-limiting aspects, for example, the dietary supplements can comprise, consist essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, or any range derivable therein, of at least one of the ingredients that are mentioned throughout the specification and claims. In non-limiting aspects, the percentage can be calculated by weight or volume of the total dietary supplement. A person of ordinary skill in the art would understand that the concentrations can vary depending on the addition, substitution, and/or subtraction of ingredients in each dietary supplement.

### IV. Method of Use

As with any dietary supplement, the effectiveness of the disclosed compositions depend on various factors, including the subject's age, intake regimen, metabolic response, genetics, gut microbiota composition, lifestyle choices, and pre-existing health conditions. Given these individual variations, the use cases presented herein should be considered as general guidance rather than definitive outcomes, as responses may differ from person to person.

In some aspects, the current disclosure also encompasses a method of managing weight and/or improving metabolic health in a subject in need thereof, the method comprising administering to the subject a dietary supplement comprising: one or more sources of anthocyanins; one or more sources of phenylpropanoids; a polyphenol blend; and at least one excipient. In some aspects, the dietary supplement comprises Hibiscus sabdariffa (rosella) flower extract; Lippia citriodora (lemon verbena) leaf extract; Cissus quadrangularis whole plant extract; a polyphenol blend; and at least one excipient. In some aspects, the composition may be for use as a dietary supplement for supporting metabolic health, healthy appetite management, and/or healthy body composition. In some aspects, the dietary supplement is formulated to naturally support GLP-1 production, help with appetite management, support healthy weight loss/body composition.

In some aspects, this disclosure provides methods for supporting healthy metabolic function in a subject, including supporting the body's natural production and activity of GLP-1. GLP-1 (Glucagon-Like Peptide-1) is a hormone produced in the gut in response to food intake, playing a key role in glucose metabolism, appetite regulation, and digestion. It binds to GLP-1 receptors (GLP-1R) found in the pancreas, brain, and gut, triggering effects that support healthy body composition. In the brain, GLP-1 reduces hunger and increases feelings of fullness by acting on the hypothalamus, leading to lower food intake. In the digestive system, it slows gastric emptying, prolonging satiety and reducing calorie consumption. Additionally, in the pancreas, GLP-1 enhances insulin secretion while suppressing glucagon release, helping to maintain healthy blood sugar levels and support metabolic health. In some aspects, the one or more ingredients in the disclosed dietary supplements may be effective in enhancing the production of GLP-1 in a subject, and/or reducing the degradation of GLP-1. In some aspects, the administering of the disclosed dietary supplement may enhance the overall levels of GLP-1 in the subject by more than or about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, or any range derivable therein, when administered for at least, at most, about 1 week, 2 weeks, 3, weeks, 4, weeks, 1 month, 2 months, 3 months, 4 months, 5 months, or 6 months. In some aspects, the levels of GLP-1 are estimated using methods well known in the art. In some aspects, the method involves estimating the amount of GLP-1 in a biological fluid sample (for example blood, serum, plasma etc.) using one or more techniques known in the art, for example ELISA (colorimetric immunoassay), RIA (radioimmunoassay), LC-MS/MS (liquid chromatography-mass spectrometry for high precision), CLIA (chemiluminescent immunoassay), or ECLIA (electrochemiluminescence for enhanced sensitivity). In some aspects, the levels may be estimated at a particular time of the day, before, or after meals as suggested by a physician or a person with skill in the art. In some aspects, administering the disclosed dietary supplement may enhance the feeling of fullness after meals by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or more, %, or any range derivable therein when administered for at least, at most, about 1 week, 2 weeks, 3, weeks, 4, weeks, 1 month, 2 months, 3 months, 4 months, 5 months, or 6 months, or any range derivable therein, as determined by a professional skilled in the art. In some aspects, the administering the disclosed dietary supplement reduces the daily calorie intake of the subject by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% or more %, or any range derivable therein when administered for at least, at most, about 1 week, 2 weeks, 3, weeks, 4, weeks, 1 month, 2 months, 3 months, 4 months, 5 months, or 6 months, or any range derivable therein.

In some aspects, the dietary supplements and methods described herein may support one or more of healthy blood glucose levels already within normal range, support healthy gastric emptying, decrease the body mass index (BMI), support healthy blood hemoglobin A1c (HbAlc) levels already within normal range, support healthy insulin function, and/or support healthy glucagon levels. In certain aspects, dietary supplements and methods of using the same described herein support the maintenance of healthy serum glucose and serum insulin levels (e.g., serum glucose and serum insulin concentrations).

### A. Administration

In some aspects, the disclosed dietary supplement may be administered in various dosage and may be suitably administered in an oral form. Moreover, the dietary supplement may exhibit an effective amount in an administration range of approximately 500 mg to 3,000 mg, for example, from about 400 mg to about 600 mg, about 500 mg to about 800 mg, about 600 mg to about 900 mg, or about 700 mg to about 1000 mg, or about 1000 mg to about 1200 mg, or about 1200 mg to about 1400 mg, or about 1400 mg to about 1600 mg, or about 1600 mg to about 1800 mg, or about 1800 mg to about 2000 mg daily. In some aspects, an effective amount may comprise a dosage of from about 1 mg/Kg to about 100 mg/Kg body weight. The dietary supplement may be administered at various doses and in various ways, for example, once a day or several times by dividing daily doses, depending on a patient's body weight, age, sex, health condition, a diet, administration time, an administration method, and the severity of a disease. In some aspects, administration of a disclosed dietary supplement as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some aspects, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. For example, the period of administration of the disclosed dietary supplement followed by a period where administration is stopped is repeated for a determined or undetermined period of time. In some aspects, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some aspects, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some aspects, the dietary supplement is for administration at least, at most, about 5 min, 10 min, 20 min, 30 min, 40 min, 50 min, 60 min, 70 min, 80 min, 90 min prior to intake of a meal (for example, breakfast, lunch, or dinner). In some aspects, the dietary supplement is for administration at least, at most, about 5 min, 10 min, 20 min, 30 min, 40 min, 50 min, 60 min, 70 min, 80 min, 90 min after intake of a meal (for example, breakfast, lunch, or dinner). In some aspects, the dietary supplement is formulated for oral intake as a once daily, twice daily, thrice daily or 4 times daily intake.

In some aspects, the subject is from two years of age to 12 years of age, or 12 years of age through 21 years of age, 21 years of age to 31 years of age, 31 years of age to 41 years of age, 41 years of age to 51 years of age, 51 years of age to 61 years of age, 61 years of age to 71 years of age, 71 years of age to 81 years of age, 81 years of age to 91 years of age, 91 years of age to 101 years of age or more. In some aspects, the subject is an adult subject. In some aspects, the subject is a non-human animal. In some aspects, the subject is a human.

### EXAMPLES

### Example 1: Materials Used

The active ingredients in Table 1 were used to make and test the disclosed dietary supplement.

TABLE 1
Ingredients

| | Amounts (per 1000 mg |
|---|---|
| | of formulation |
| | excluding excipients) |
| Hibiscus sabdariffa (flower) and Lippia | 500 mg |
| citriodora (leaf) supplied by Suannutra under | |
| the tradename METABOLAID ® | |
| Cissus quadrangularis (stem and leaves) | 300 mg |
| extract, supplied by Gateway Health Alliance | |
| under the tradename Cissus CQR-300 ®. | |
| Green tea (Camellia sinensis), pomegranate | 200 mg |
| (Punica granatum), and grape (Vitis vinifera) | |
| whole plant extract, supplied by Vidya Herbs | |
| under the tradename Polyphenol Blend. | |

Additional ingredients include rice flour, vegetable capsule (hypermellose), silicon dioxide, and magnesium stearate.

What is claimed is:

1. A dietary supplement comprising:
Hibiscus sabdariffa (rosella) flower extract;
Lippia citriodora (lemon verbena) leaf extract;
Cissus quadrangularis whole plant extract;
a polyphenol blend; and
at least one excipient.

2. The dietary supplement of claim 1, wherein the dietary supplement comprises at least, about, or at most 1-10% by weight of rosella flower extract, and at least, about, or at most 1-30% by weight lemon verbena leaves extract.

3. The dietary supplement of claim 2, wherein the Hibiscus sabdariffa flower and the Lippia citriodora leaf extracts are present in a 2:0.5 to 0.5:2 ratio by weight.

4. The dietary supplement of claim 3, wherein the dietary supplement comprises Metabolaid®.

5. The dietary supplement of claim 1, wherein the polyphenol blend provides at least, about, or at most 100-300 mg of polyphenols per 1000 mg of the dietary supplement excluding excipients.

6. The dietary supplement of claim 1, wherein the polyphenol blend comprises green tea (Camellia sinensis), pomegranate (Punica granatum), and grape (Vitis vinifera) whole plant extract.

7. The dietary supplement of claim 1, wherein the at least one excipient comprises one or more diluents, stabilizers, antioxidants, fillers, carriers, adjuvants, lubricant, preserving agents, stabilizing agents, wetting agents, emulsifying agents, solution promoters, salts, solubilizers, antifoaming agents, antioxidants, dispersing agents, surfactants, or colorants, or any combination thereof.

8. The dietary supplement of claim 7, wherein the filler is rice flour, carrier is Hypromellose, the dispersing agent is silicon dioxide, and/or the lubricant is magnesium stearate.

9. (canceled)

10. (canceled)

11. (canceled)

12. The dietary supplement of claim 1, wherein the dietary supplement comprises about 300-700 mg combined of Hibiscus sabdariffa and Lippia citriodora extracts, about 200-400 mg of Cissus quadrangularis extract, and about 200-400 mg polyphenol blend per 1000 mg of the dietary supplement excluding excipients.

13. The dietary supplement of claim 12, comprising:
about 500 mg by weight of Hibiscus sabdariffa flower and Lippia citriodora leaf extract per 1000 mg of the dietary supplement excluding excipient weight;
about 300 mg by weight of Cissus quadrangularis extract per 1000 mg of the dietary supplement excluding excipient weight;
about 200 mg by weight of polyphenol blend per 1000 mg of the dietary supplement excluding excipient weight;
and at least one excipient.

14. The dietary supplement of claim 1, wherein the dietary supplement is in the form of a solid, a semi-solid, or a liquid.

15. The dietary supplement of claim 14, wherein the dietary supplement is in the form of a tablet, a capsule, or a gummy.

16. The dietary supplement of claim 1, wherein the dietary supplement is free from one or more of artificial colors, and preservatives, gluten, soy, nut, and dairy.

17. (canceled)

18. The dietary supplement of claim 1, wherein the dietary supplement is vegan.

19. The dietary supplement of claim 1, wherein administering the dietary supplement to a subject in need thereof, enhances the production of glucagon-like peptide 1 (GLP-1) in the subject, reduces the degradation of glucagon-like peptide 1 (GLP-1) in the subject, and/or increases the overall levels of GLP-1 by over 30% in the first 8 weeks of daily administration.

20. (canceled)

21. (canceled)

22. The dietary supplement of claim 19, wherein the subject is a human.

23. A method of managing weight and/or improving metabolic health in a subject in need thereof, the method comprising administering to the subject a dietary supplement comprising:
one or more sources of anthocyanins;
one or more sources of phenylpropanoids;
a polyphenol blend; and
at least one excipient.

24. The method of claim 23, wherein the method comprises administering the dietary supplement in an amount that provides about 500 mg of Metabolaid®, 300 mg of Cissus extract and 200 mg of the polyphenol blend daily, at least 15-45 minutes before a meal.

25. The method of claim 24, wherein the amount is administered in a single dose, 2 doses, or more.

26. The method of claim 23, wherein the method comprises administering the dietary supplement orally.

27. (canceled)

28. (canceled)

29. (canceled)

30. A method of administering a dietary supplement to a subject, the method comprising:
a. providing a dietary supplement as claimed in claim 1; and
b. instructing the subject to take 2 capsules daily, 30 minutes before a meal.
